(19) United States
(12) Patent Application Publication
(10) Pub. No.: US 2009/0054753 A1
(43) Pub. Date: Feb. 26, 2009

(54) Variable Sampling Interval for Blood Analyte Determinations

(76) Inventors: Mark Ries Robinson, Albuquerque, NM (US); Mike Borrello, Carlsbad, CA (US); Dave McMahon, Solana Beach, CA (US)

Correspondence Address: V. Gerald Grafe, esq., P.O. Box 2689, Corrales, NM, 87048, US

(21) Appl. No.: 11/842,624

(22) Filed: Aug. 21, 2007

(51) Int. Cl. A61B 5/00; G01N 33/50

(52) U.S. Cl. 600/365; 422/68.1

(57) ABSTRACT

The present invention provides methods and apparatuses that can provide measurement of glucose with variable intervals between measurements, allowing more efficient measurement with greater patient safety. A method according to the present invention can comprise measuring the value of an analyte such as glucose at a first time; determining a second time from a patient condition, an environmental condition, or a combination thereof; then measuring the value of the analyte at the second time (where the second time can be expressed as an interval after the first time, an absolute time, or a time indicated when certain patient or environmental conditions, or both, are reached or detected). The second time can be determined, as an example, from a comparison of the analyte value at the first time with a threshold. The interval between the first time and the second time can be related to the difference between the analyte value at the first time and the threshold; e.g., the closer to the threshold, the closer the two measurement times. The invention can be used with automated measurement systems, allowing the system to determine measurement times and automatically make measurements at the determined times, reducing operator interaction and operator error.

### CROSS-REFERENCE TO RELATED APPLICATIONS

This application is related to U.S. provisional application 60/791,719, filed Apr. 12, 2006, and to U.S. provisional application 60/737,254, filed Nov. 15, 2006, each of which is incorporated herein by reference.

### FIELD OF THE INVENTION

This invention relates to the field of the measurement of blood analytes, and more specifically to multiple measurements of analytes such as glucose in blood that has been temporarily or permanently removed from a body.

### BACKGROUND OF THE INVENTION

Many peer-reviewed publications have demonstrated that tight control of blood glucose significantly improves critical care patient outcomes. Tight glycemic control (TGC) has been shown to reduce surgical site infections by 60% in cardiothoracic surgery patients and reduce overall ICU mortality by 40% with significant reductions in ICU length of stay. See, e.g., Furnary Tony, Oral presentation at 2005 ADA annual, session titled “Management of the Hospitalized Hyperglycemic Patient;” Van den Berghe et al., NEJM 2001; 345:1359. Historically, caregivers have treated hyperglycemia (high blood glucose) only when glucose levels exceeded 220 mg/dl. Based upon recent clinical findings, however, experts now recommend IV insulin administration to control blood glucose to within the normoglycemic range (80-110 mg/dl). Adherence to such strict glucose control regimens requires frequent monitoring of blood glucose and frequent adjustment of insulin infusion to achieve normoglycemia while avoiding risk of hypoglycemia (low blood glucose). In response to the demonstrated clinical benefit, approximately 50% of US hospitals have adopted some form of tight glycemic control with an additional 23% expected to adopt protocols within the next 12 months. Furthermore, 36% of hospitals already using glycemic management protocols in their ICUs plan to expand the practice to other units and 40% of hospitals that have near-term plans to adopt TGC protocols in the ICU also plan to do so in other areas of the hospital. As research continues to show the benefits of driving patient's blood glucose levels even lower these tight glycemic control protocols have become increasingly labor intensive and complicated. Typical protocols today call for 44 blood glucose samples taken over a patient's 3 day stay in the ICU. Krinsley et al. have shown additional reductions in infections by maintaining down to a blood glucose levels in the 80 to 90 mg/dl range.

Given the compelling evidence for improved clinical outcomes associated with tight glycemic control, hospitals are under pressure to implement TGC as the standard of practice for critical care and cardiac surgery patients. Clinicians and caregivers have developed TGC protocols that use IV insulin administration to maintain normal patient glucose levels. To be safe and effective, these protocols require frequent blood glucose monitoring. Currently, these protocols involve periodic removal of blood samples by nursing staff and testing on handheld meters or blood gas analyzers. Although hospitals are responding to the identified clinical need, adoption has been difficult with current technology due to two principal reasons.

Fear of hypoglycemia. The target glucose range of 80-110 mg/dl brings the patient near clinical hypoglycemia (blood glucose less than 50 mg/dl). Patients exposed to hypoglycemia for greater than 30 minutes have significant risk of neurological damage. IV insulin administration with only intermittent glucose monitoring (typically hourly by most TGC protocols) exposes patients to increased risk of hypoglycemia. In a recent letter to the editors of Intensive Care medicine, it was noted that 42% of patients treated with a TGC protocol in the UK experienced at least one episode of hypoglycemia. See, e.g., lain Mackenzie et al., “Tight glycaemic control:a survey of intensive care practice in large English Hospitals;” Intensive Care Med (2005) 31:1136. In addition, handheld meters require procedural steps that are often cited as a source of measurement error, further exacerbating the fear (and risk) of accidentally taking the blood glucose level too low. See, e.g., Bedside Glucose Testing systems, CAP today, April 2005, page 44.

Burdensome procedure. Currently most tight glycemic control protocols utilize fixed sampling periods. Existing protocols are typically designed with a sampling period of every 30 minutes upon admission to the intensive care unit progressing to one hour intervals as the patient stabilizes. The procurement of the blood glucose measurement is made by a manual process. Some protocols call for an increase in sampling frequency if the patient's glucose falls outside the target range. Using current technology, each measurement requires removal of a blood sample, performance of the blood glucose test, evaluation of the result, determination of the correct therapeutic action, and finally adjustment to the insulin infusion rate. Long intervals between measurements can cause a lose of tight glycemic control, or place the patient at risk. Short time intervals between measurements place significant strain on limited ICU nursing resources that already struggle to meet patient care needs.

As used herein, a “glucose sensor” is a noncontact glucose sensor, a contact glucose sensor, or any other instrument or technique that can determine the glucose presence or concentration in a sample. As used herein, a “contact glucose sensor” is any measurement device that makes physical contact with the fluid containing the glucose under measurement. Standard glucose meters are an example of a contact glucose sensor. In use a drop of blood is placed on a disposable strip for the determination of glucose. An example of a glucose sensor is an electrochemical sensor. An electrochemical sensor is a device configured to detect the presence and/or measure the level of analyte in a sample via electrochemical oxidation and reduction reactions on the sensor. These reactions are transduced to an electrical signal that can be correlated to an amount, concentration, or level of analyte in the sample. Another example of a glucose sensor is a microfluidic chip or micro post technology. These chips are a small device with micro-sized posts arranged in varying numbers on a rectangle array of specialized material which can measure chemical concentrations. The tips of the microposts can be coated with a biologically active layer capable of measuring concentrations of specific lipids, proteins, antibodies, toxins and sugars. Microposts have been made of Foturan, a photo defined glass. Another example of a glucose sensor is a fluorescent measurement technology. The system for measurement is composed of a fluorescence sensing device consisting of a light source, a detector, a fluorophore (fluorescence dye), a quencher and an optical polymer matrix. When excited by light of appropriate wavelength, the fluorophore emits light (fluorescence). The intensity of the light or extent of quenching is dependent on the concentration of the compounds in the media. Another example of a glucose sensor is an enzyme based monitoring system that includes a sensor assembly, and an outer membrane surrounding the sensor. Generally, enzyme based glucose monitoring systems use glucose oxidase to convert glucose and oxygen to a measurable end product. The amount of end product produced is proportional to the glucose concentration. Ion specific electrodes are another example of a contact glucose sensor.

As used herein, a “noncontact glucose sensor” is any measurement method that does not require physical contact with the fluid containing the glucose under measurement. Example noncontact glucose sensors include sensors based upon spectroscopy. Spectroscopy is a study of the composition or properties of matter by investigating light, sound, or particles that are emitted, absorbed or scattered by the matter under investigation. Spectroscopy can also be defined as the study of the interaction between light and matter. There are three types of spectroscopy in widespread use: absorption spectroscopy, emission spectroscopy, and scattering spectroscopy. Absorbance spectroscopy uses the range of the electromagnetic spectrum in which a substance absorbs. After calibration, the amount of absorption can be related to the concentration of various compounds through the Beer-Lambert law. Emission spectroscopy uses the range of the electromagnetic spectrum in which a substance radiates. The substance first absorbs energy and then radiates this energy as light. This energy can be from a variety of sources including collision and chemical reactions. Scattering spectroscopy estimates certain physical characteristics or properties by measuring the amount of light that a substance scatters at certain wavelengths, incidence angles and polarization angles. One of the most useful applications of light scattering spectroscopy is Raman spectroscopy but polarization spectroscopy has also been used for analyte measurements.

The list below describes several types of spectroscopy, but should not be considered an exhaustive list. Atomic Absorption Spectroscopy is where energy absorbed by the sample is used to assess its characteristics. Sometimes absorbed energy causes light to be released from the sample, which may be measured by a light sensing technique such as fluorescence spectroscopy. Attenuated total reflectance spectroscopy is used to sample liquids where the sample is penetrated by an energy beam one or more times and the reflected energy is analyzed. Attenuated total reflectance spectroscopy and the related technique called frustrated multiple internal reflection spectroscopy are used to analyze liquids. Electron Paramagnetic Spectroscopy is a microwave technique based on splitting electronic energy fields in a magnetic field. It is used to determine structures of samples containing unpaired electrons. Electron Spectroscopy includes several types of electron spectroscopy, all associated with measuring changes in electronic energy levels. Gamma-ray Spectroscopy uses Gamma radiation as the energy source in this type of spectroscopy, which includes activation analysis and Mossbauer spectroscopy. Infrared Spectroscopy uses the infrared absorption spectrum of a substance, sometimes called its molecular fingerprint. Although frequently used to identify materials, infrared spectroscopy also is used to quantify the number of absorbing molecules.

Some types of spectroscopy include the use of mid-infrared light, near-infrared light and uv/visible light. Fluorescence spectroscopy uses photons to excite a sample which will then emit lower energy photons. This type of spectroscopy has become popular in biochemical and medical applications. It can be used with confocal microscopy, fluorescence resonant energy transfer, and fluorescent lifetime imaging. Laser illumination can be used with many spectroscopic techniques to include absorption spectroscopy, fluorescence spectroscopy, Raman spectroscopy, and surface-enhanced Raman spectroscopy. Laser spectroscopy provides information about the interaction of coherent light with matter. Laser spectroscopy generally has high resolution and sensitivity. Mass spectrometry uses a mass spectrometer source to produce ions. Information about a sample can be obtained by analyzing the dispersion of ions when they interact with the sample, generally using the mass-to-charge ratio. Multiplex or Frequency-Modulated Spectroscopy is a type of spectroscopy where each optical wavelength that is recorded is encoded with a frequency containing the original wavelength information. A wavelength analyzer can then reconstruct the original spectrum. Hadamard spectroscopy is another type of multiplex spectroscopy. Raman spectroscopy uses Raman scattering of light by molecules to provide information on a sample's chemical composition and molecular structure. X-ray Spectroscopy is a technique involving excitation of inner electrons of atoms, which may be seen as x-ray absorption. An x-ray fluorescence emission spectrum can be produced when an electron falls from a higher energy state into the vacancy created by the absorbed energy. Nuclear magnetic resonance spectroscopy analyzes certain atomic nuclei to determine different local environments of hydrogen, carbon and other atoms in a molecule of an organic compound. Grating or dispersive spectroscopy typically records individual groups of wavelengths. As can be seen by this brief survey, there are multiple methods and means of spectroscopic techniques that can be applied to measuring analytes such as glucose.

Glucose measurements can be made in various media. Types of glucose measurements represented in the media include ISF microdialysis sampling and online measurements, continuous alternate site measurements, ISF fluid measurements, tissue glucose measurements, ISF tissue glucose measurements, body fluid measurements, skin measurement, skin glucose measurements, subcutaneous glucose measurements, extracorporeal glucose sensors, in-vivo glucose sensors, and ex-vivo glucose sensors. Examples of such systems include those described in U.S. Pat. No. 6,990,366 Analyte Monitoring Device and Method of Use; U.S. Pat. No. 6,259,937 Implantable Substrate Sensor; U.S. Pat. No. 6,201,980 Implantable Medical Sensor System; U.S. Pat. No. 6,477,395 Implantable in Design Based Monitoring System Having Improved Longevity Due to in Proved Exterior Surfaces; U.S. Pat. No. 6,653,141 Polyhydroxyl-Substituted organic Molecule Sensing Method and Device; US patent application 20050095602 Microfluidic Integrated Microarrays For Biological Detection; each of the preceding incorporated by reference herein.

The many types of glucose sensors and glucose sensing systems that have been proposed present a range of tradeoffs. The problem of effectively integrating glucose measurements into current patient care practices remains important, however, regardless of which sensor or system is used.

### SUMMARY OF THE INVENTION

The present invention comprises methods and apparatuses that can provide measurement of glucose with variable intervals between measurements, allowing more efficient measurement with greater patient safety. A method according to the present invention can comprise measuring the value of an analyte such as glucose at a first time; determining a second time from a patient condition, an environmental condition, or a combination thereof; then measuring the value of the analyte at the second time (where the second time can be expressed as an interval after the first time, an absolute time, or a time indicated when certain patient or environmental conditions, or both, are reached or detected). The second time can be determined, as an example, from a comparison of the analyte value at the first time with a threshold. The interval between the first time and the second time can be related to the difference between the analyte value at the first time and the threshold; e.g., the closer to the threshold, the closer the two measurement times. The invention can be used with automated measurement systems, allowing the system to determine measurement times and automatically make measurements at the determined times, reducing operator interaction and operator error.

In other example embodiments, the second time can be determined from a prediction of the value of the analyte. For example, the patient's conditions or environmental conditions, or both, can be used to predict a time at which the analyte level will reach a threshold, and the second time be determined to be that predicted time. A safety margin can be imposed on the threshold, or the time, or both, if desired. The prediction of the time can be based on linear or non-linear extrapolation from previous analyte values. The mechanism for determining the next sampling time can be based on a physiological model of the patient. It can also consider information related to infusion of nutrients, insulin, glucose, or other substances. Certain changes in patient or environmental conditions can also be used to indicate that a measurement be made; e.g., a glucose measurement can be automatically initiated when a change in glucose infusion rate is made.

In some embodiments of the present invention, a second measurement can be made when a physiologic model of the patient, considering patent conditions, environmental conditions, or a combination, predicts a glucose level that has reached a threshold value. Both high and low thresholds can be established, with symmetric or asymmetric safety margins if desired. Example physiologic models suitable for use in the present invention can include a Netter diagram model, AIDA model (http://www.2aida.net/welcome/, visited Sep. 16, 2007, incorporated herein by reference), Chase model, Bergman model, compartment model with differential equations, insulin pharmacokinetics and distribution model, glucose pharmacokinetics and distribution model, meal model, glucose/insulin pharmacodynamic model, and insulin secretion and kinetics model, or a combination of two or more of the preceding. A model can be applied and a second time determined as of the preceding measurement, or the model can be updated as time lapses or patient or environmental conditions change. The model can be adjusted to better fit the patient by considering previous combinations of patient and environmental conditions and measured analyte values.

Some embodiments of the present invention can use an optical measurement of analyte in whole blood. Some embodiments of the present invention can use measurements of analyte in portions of blood samples after removal of substantially all the red blood cells in the portion.

The present invention also provides apparatuses useful for determining analyte values such as blood glucose concentrations. Such apparatuses can comprises a fluid access system, adapted to withdraw a sample of a bodily fluid such as blood from a patient; an analyte measurement system, adapted to measure the value of an analyte such as glucose concentration from the blood sample; and a controller, adapted to cause the fluidics system to withdraw a fluid sample for measurement at times determined by patient conditions, environmental conditions, or a combination thereof.

Advantages and novel features will become apparent to those skilled in the art upon examination of the following description or can be learned by practice of the invention. The advantages of the invention can be realized and attained by means of the methods, example embodiments, and combinations specifically described in the disclosure and in the appended claims.

### BRIEF DESCRIPTION OF THE DRAWINGS

FIG. 1 is a schematic illustration of the present invention in use with a patient.

FIG. 2 is a schematic illustration of the present invention in use with a patient.

FIG. 3(a,b,c) is a schematic illustration of the operation of an example embodiment of the present invention.

FIG. 4 is a Netter physiological response diagram illustrating interactions governing glucose consumption and production.

FIG. 5 is a block diagram of interactions governing glucose consumption and production.

FIG. 6 is a presentation of equations governing the Chase et al. model as well as the input parameters.

FIG. 7 is a state diagram of the Chase model showing inputs and relationships of the model.

FIG. 8 is a schematic illustration of an example of using a physiological model such as the Chase model as an estimator of glucose concentration and the use of such an estimate to determine a next measurement time.

FIG. 9 is a graphical representation of automated determination of a next measurement time.

FIG. 10 is a schematic illustration of an example embodiment of the present invention.

FIG. 11 is a schematic illustration of an example embodiment of the present invention in operation with an automated blood removal system

### DETAILED DESCRIPTION OF THE INVENTION

The present invention comprises methods and apparatuses that can provide measurement of glucose with variable intervals between measurements, allowing more efficient measurement with greater patient safety. A method according to the present invention can comprise measuring the value of an analyte such as glucose at a first time; determining a second time from a patient condition, an environmental condition, or a combination thereof; then measuring the value of the analyte at the second time (where the second time can be expressed as an interval after the first time, an absolute time, or a time indicated when certain patient or environmental conditions, or both, are reached or detected). The second time can be determined, as an example, from a comparison of the analyte value at the first time with a threshold. The interval between the first time and the second time can be related to the difference between the analyte value at the first time and the threshold; e.g., the closer to the threshold, the closer the two measurement times. The invention can be used with automated measurement systems, allowing the system determine measurement times and automatically make measurements at the determined times, reducing operator interaction and operator error.

In other example embodiments, the second time can be determined from a prediction of the value of the analyte. For example, the patient's conditions or environmental conditions, or both, can be used to predict a time at which the analyte level will reach a threshold, and the second time be determined to be that predicted time. A safety margin can be imposed on the threshold, or the time, or both, if desired. The prediction of the time can be based on linear or non-linear extrapolation from previous analyte values. The mechanism for determining the next sampling time can be based upon a physiological model of the patient. It can also consider information related to infusion of nutrients, insulin, glucose, or other substances. Certain changes in patient or environmental conditions can also be used to indicate that a measurement be made; e.g., a glucose measurement can be automatically initiated when a change in glucose infusion rate is made.

In some embodiments of the present invention, a second measurement can be made when a physiologic model of the patient, considering patent conditions, environmental conditions, or a combination, predicts a glucose level that has reached a threshold value. Both high and low thresholds can be established, with symmetric or asymmetric safety margins if desired. Example physiologic models suitable for use in the present invention can include a Netter diagram model, AIDA model, Chase model, Bergman model, compartment model with differential equations, insulin pharmacokinetics and distribution model, glucose pharmacokinetics and distribution model, meal model, glucose/insulin pharmacodynamic model, and insulin secretion and kinetics model, or a combination of two or more of the preceding. A model can be applied and a second time determined as of the preceding measurement, or the model can be updated as time lapses or patient or environmental conditions change. The model can be adjusted to better fit the patient by considering previous combinations of patient and environmental conditions and measured analyte values.

Some embodiments of the present invention can use an optical measurement of analyte in whole blood. Some embodiments of the present invention can use measurements of analyte in portions of blood samples after removal of substantially all the red blood cells in the portion.

The present invention also provides apparatuses useful for determining analyte values such as blood glucose concentrations. Such apparatuses can comprises a fluid access system, adapted to withdraw a sample of a bodily fluid such as blood from a patient; an analyte measurement system, adapted to measure the value of an analyte such as glucose concentration from the blood sample; and a controller, adapted to cause the fluidics system to withdraw a fluid sample for measurement at times determined by patient conditions, environmental conditions, or a combination thereof.

The present invention comprises methods and apparatuses that can provide measurement of analytes such as glucose at intervals determined based on characteristics of the patient. Varying the sampling interval based on the patient's condition can allow close control of the patient's glucose without requiring an excessive number of measurements. A glucose measurement can be made, and a “next-sample-condition” defined based on environmental conditions (e.g., ventilation state, infusion rates, etc.), the patient's condition (e.g., recent glucose level, past response, etc.), or a combination thereof. When the next-sample-condition is satisfied, then a subsequent glucose measurement can be made. Using such a next-sample-condition allows the number of samples taken to be reduced while still maintaining tight and safe control of an analyte such glucose.

As used in connection with the present invention, “the patient's condition” or “patient condition” includes without limitation parameters of the patient such as physiological parameters like blood pressure, previous glucose measurements; previous response to glucose or insulin or medication or other treatment; presence, stage, or type of diabetes, other physical conditions; previous responses to the preceding or to environmental conditions. As used in connection with the present invention, “environmental conditions” includes without limitation controlled parameters such as medication or nutrient infusion rates, state of other treatments such as ventilators; temperature or humidity.

The present invention is particularly useful in combination with a measurement system that can automatically measure glucose, for example such as those described in U.S. patent application Ser. No. 11/352,956 “Apparatus and methods for analyzing body fluid samples”, filed Feb. 13, 2006; Ser. No. 11/316,407 “Apparatus and methods for analyzing body fluid samples”, filed Dec. 21, 2005; Ser. No. 10/850,646 “Analyte determinations”, filed May 21, 2004; Ser. No. 11/679,826 “Blood Analyte Determinations”, filed Feb. 27, 2007; Ser. No. 11/679,837 “Analyte Determinations”, filed Feb. 28, 2007; Ser. No. 11/679,839 “Analyte Determinations”, filed Feb. 28, 2007; Ser. No. 11/679,835 “Analyte Determinations”, filed Feb. 27, 2007; each of which is incorporated herein by reference. Such systems, combined with the present invention, can provide measurements whose frequency is adjusted to meet clinical requirements. By automatically determining the sampling time and by having the ability to procure a blood glucose measurement automatically, the system can ensure the time period associated with undetected hyper or hypo glycemia is minimized. As the patient becomes likely to approach the target glucose limits, the system increases its sampling frequency such that the time a patient spends outside of the target zone without a glucose measurement to allow corrective action is minimized. The ability of the system to both determine the next sampling time as well as perform a measurement automatically results in a system that is safer than a system totally dependent upon manual intervention by the care provider for each measurement.

Utilization of a measurement frequency greater than required for sufficient control results in a measurement rate that can be undesirable, as well. Generally, there is some cost or risk associated with each measurement event. As examples, many measurement systems require some patient blood loss for each measurement, so too frequent measurements can lead to undesirable blood loss. Some measurement systems result in saline infused into the patient with each measurement, so too frequent measurements can lead to undesirable blood dilution with saline. Some measurement systems require saline to clean or flush parts of the system, so too frequent measurements can cause added expense associated with consumption and replacement of saline and disposing of waste. Some measurement systems require disposable strips or enzymes for each measurement, so too frequent measurements can cause added expense associated with consumption of strips or enzymes. Exposure of the blood access system to blood products can risk aggregating, clotting, or system occlusions, so too frequent measurements can increase the risk of an adverse occurrence. Accessing a blood sample for measurement can risk infection, so too frequent measurements can increase the overall risk of infection. In current clinical practice the cost and risk associated with obtaining a glucose measurement is high, so in some hospitals measurements are made less frequently than desirable resulting in compromised patient care and safety. However, the risk of poor glucose control is known, so in other conditions measurements can be made more frequently than required for patient care and safety resulting in the risks described above.

A patient's systemic glucose value and the rate of change of the systemic glucose value result from a complex interaction among many internal and external factors. The determination of the next measurement time can rely on any of, or a combination of, factors such as the following.

Glucose level: as the patient begins to approach the blood glucose target limits the rate of sampling can increase such the time outside this target range is minimized. The glucose level can be utilized as a parameter to determine the next sampling time.

Rate of glucose change: if the patient's blood glucose is changing rapidly the glucose may quickly exceed a target limit. The rate of glucose change can be utilized as a parameter to determine the next sampling time.

Insulin dosing history: the insulin dosing history will influence the expected rate of change and the level of blood glucose. Insulin dosing history can be utilized as a parameter to determine the next sampling time.

Caloric intake history: the caloric intake history will influence the expected change and magnitude of the blood glucose. Changes in the amount of calories administered, or rate at which calories are administered, to the patient either by mouth or via the blood system can be utilized as a parameter to determine the next sampling time.

Medications: medications can influence the body's regulation of blood glucose and response to insulin. Medication information can be utilized as a parameter to determine the next sampling time.

Insulin sensitivity: insulin sensitivity is a general measure of the body's response to insulin dosing. This factor can change as the patient's physiological status changes and can be useful in determining the patient's response to therapy. The patient's insulin sensitivity can be determined in various ways, for example by input from a care provider, by inference from other conditions, or by determination from previous insulin dosing and glucose measurement information. Insulin sensitivity can be utilized as a parameter to determine the next sampling time.

Target glucose range: the lower and tighter the range the more difficult it can be to maintain the patient's blood glucose level within this target range. The target glucose range can be utilized as a parameter to determine the next sampling time.

Duration of time in the intensive care unit: upon admission to the intensive care unit most patients will have a high glucose level with an initial therapy goal of getting the patient in the target range. This period is typically one with high rates of glucose change and can require more frequent monitoring. Information regarding the duration of time in the intensive care unit can be utilized as a parameter to determine the next sampling time.

Model based parameters, estimated states and state predictions: The response of the glucose level to the factors noted above can be mathematically modeled to estimate model parameters and states. The estimated parameters of this model (including insulin sensitivity) can be utilized to determine the next sampling time.

The next sampling time can be determined as an interval from the previous sampling time. For example, the invention can determine that the next glucose measurement should be made 30 minutes after the preceding measurement. The measurement system can simply wait until 30 minutes have passed and then perform the measurement. The next sampling time can also be determined based on patient conditions or environmental conditions as they change. For example, the invention can determine that the next glucose measurement should be made within 10 minutes of when the insulin infusion rate changes. The next sampling time can also be determined by a combination of the above methods, so that the time since last glucose measurement is a parameter to be considered along with other parameters. For example, the invention can determine that the next glucose measurement should be made 45 minutes after the preceding measurement, but an intervening parameter chance (e.g., nutrient infusion rate change) can indicate an earlier or later time for the next measurement. The next sampling time can be determined to be a time that will provide a glucose measurement before the patient's glucose is anticipated to be outside of a target range, allowing for adjustments of therapy to maintain the desired glucose value.

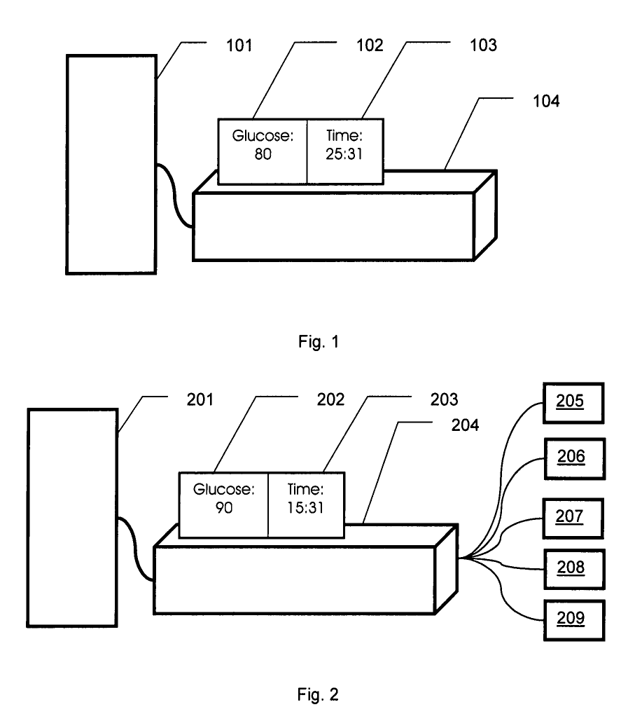

FIG. 1 is a schematic illustration of the present invention in use with a patient. A glucose measurement system 104 is in communication with a patient 101. The glucose measurement system provides an indication 102 of the patient's glucose level. The present invention provides an indication 103 of the time remaining until the next glucose measurement should be made. Placing the measurement time determination in communication with the glucose measurement system 104 can be efficient by eliminating any need to manually enter glucose measurement results. The glucose measurement system 104 can be, as examples, systems such as those described in U.S. Patent applications (blood access system applications). The glucose measurement system can also comprise other manual or automated measurement systems, as an example a conventional strip-based glucose meter conveniently placed in data communication with an apparatus implementing a method according to the present invention. A method according to the present invention can be implemented in a standalone processing system, placed in communication with the glucose measurement system and any other information sources necessary for the determination of the next measurement time. It can also be implemented as part of the glucose measurement system, taking advantage of efficient data communication and control. For example, past glucose measurements can be easily communicated in such an integrated system. Also, the present invention can automatically control the glucose measurement system to take measurements at the determined times.

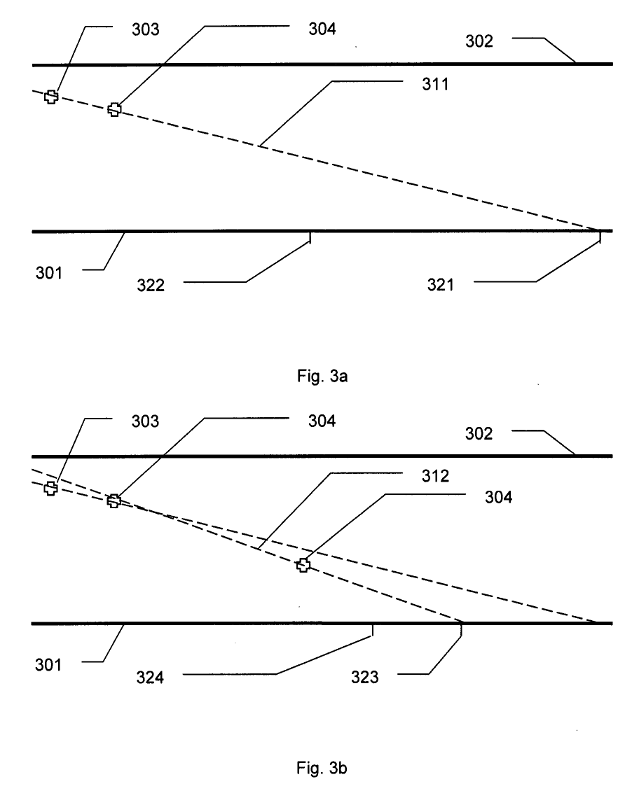

FIG. 2 is a schematic illustration of the present invention in use with a patient. A glucose measurement system 204 is in communication with a patient 201. The glucose measurement system provides an indication 202 of the patient's glucose level. The present invention provides an indication 203 of the time remaining until the next glucose measurement should be made. Placing the measurement time determination in communication with the glucose measurement system 204 can be efficient by eliminating any need to manually enter glucose measurement results. The glucose measurement system is also in data communication with systems or sensors associated with medication type and rate 205, insulin infusion 206, nutrient infusion 207, environmental conditions 208, and treatment objectives 209. The glucose measurement system 204 can be, as examples, systems such as those described in U.S. Patent applications (blood access system applications). The glucose measurement system can also comprise other manual or automated measurement systems, as an example a conventional strip-based glucose meter conveniently placed in data communication with an apparatus implementing a method according to the present invention. A method according to the present invention can be implemented in a standalone processing system, placed in communication with the glucose measurement system and the other information sources necessary for the determination of the next measurement time. It can also be implemented as part of the glucose measurement system, taking advantage of efficient data communication and control. For example, past glucose measurements can be easily communicated in such an integrated system. Also, the present invention can automatically control the glucose measurement system to take measurements at the determined times.

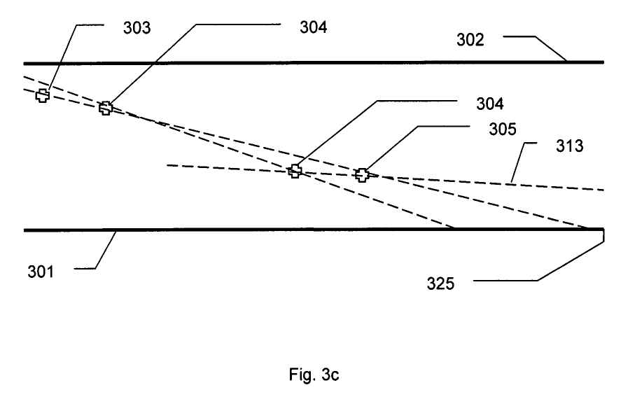

A method according to the present invention can determine a measurement time based only on past glucose measurements and target glucose range. FIG. 3(a,b,c) is a schematic illustration of the operation of such an example embodiment. The target glucose range is depicted by horizontal lines 301, 302, with time depicted as advancing from left to right in the figure. In FIG. 3a, two past glucose measurement values 303, 304 are used to determine by straight line interpolation 311 an expected time 321 at which the patient's glucose will reach one of the boundaries of the range. A next measurement time 322 can be determined by applying a safety margin to the expected time 321.

In FIG. 3b, a measurement 304 has been taken at the time indicated in FIG. 3a. Straight line interpolation 312 can be used to determine a new expected time 323, and a next measurement time 324 determined by applying a safety factor to that expected time. In FIG. 3c, a measurement 305 has been taken at the time indicated in FIG. 3b. Straight line interpolation 313 yields an expected time that is beyond a maximum measurement interval, so the system determines a next measurement time 325 as the maximum measurement interval.

While only two measurements and straight line interpolation were used in the discussion of FIG. 3(a,b,c) for simplicity of illustration, additional measurement values and more complex techniques (e.g., curve fitting techniques known to those skilled in the art) can be used to determine measurement times. For example, multiple past measurements can be used as inputs to polynomial curve fitting methods, autoregressive methods, moving average methods, and proportional derivative methods. Also, the target range can be variable, for example corresponding to changes in desired treatment characteristics. Also, multiple patient conditions or environmental conditions can be used in determining the next measurement time, allowing the method to adjust for changes in glucose measurement as well as changes in conditions such as infusion rate of one or more substances, ventilator status, etc.

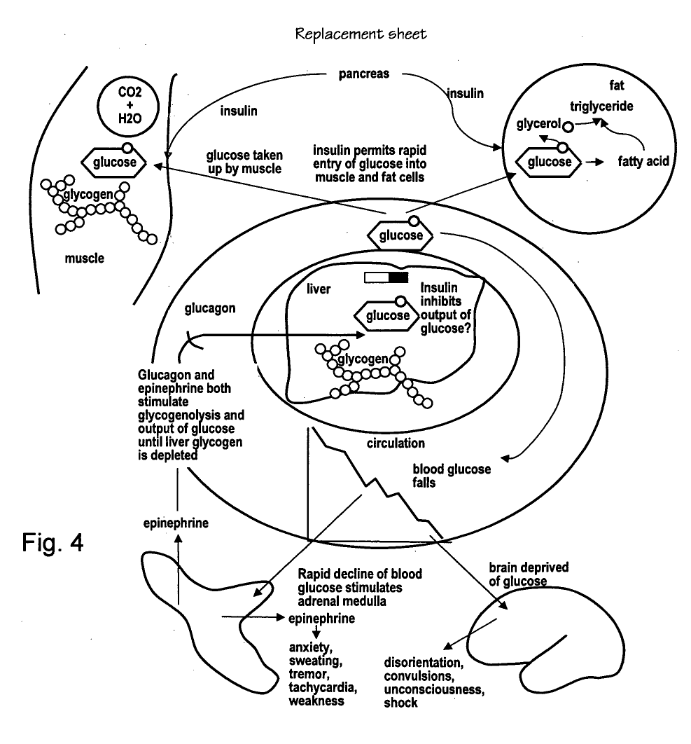

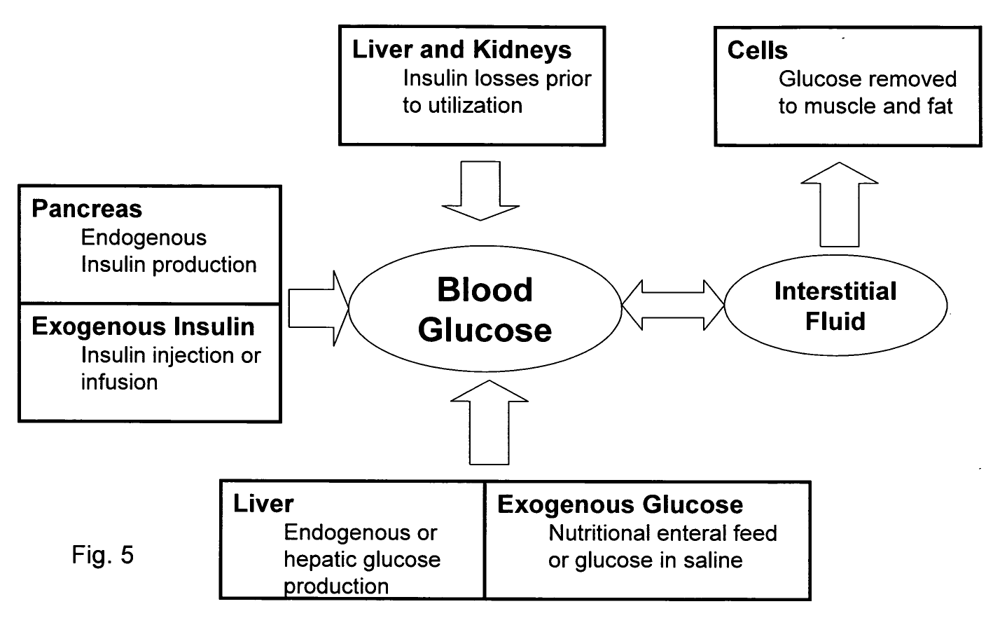

The determination of a next measurement time, described previously in the context of mathematical determinations based on previous values, can also be based on a physiological model of the patient's response to patient conditions, environmental conditions, or a combination thereof. FIG. 4 is a Netter physiological response diagram showing the main interactions governing glucose consumption and production. A block diagram of these interactions is shown in FIG. 5. As illustrated in FIG. 5, blood glucose is affected by endogenous insulin produced by the pancreas and exogenous insulin supplied by injection or infusion. The liver and kidneys can provide insulin losses prior to utilization by the body. Glucose in the interstitial fluid can be removed to muscle and fat cells. Glucose can be produced in the liver and can be supplied by enteral feed or glucose infusate. There is an extensive body of literature on the physiological modeling of glucose consumption and production. Examples of these models are the AIDA model, the Minimal Model of Bergman et al., the Hovarka model and the Chase et al. model. A excellent overview of metabolic modeling in its entirety is by Carson and Cobelli, Modeling Methodology for Physiology and Medicine, Academic Press, San Diego, 2001, incorporated herein by reference. Also, basic compartment modeling with differential equations, insulin pharmacokinetics and distribution modeling, glucose pharmacokinetics and distribution modeling, meal modeling, glucose/insulin pharmacodynamic modeling, and insulin secretion and kinetics modeling can also be suitable. See, e.g., “Model-based glycaemic control in critical care—A review of the state of the possible”, Biomedical Signal Processing and Control 1 (2006) 3-21, Chase et al., incorporated herein by reference. Patient conditions and environmental conditions can be input to such a model, and the time at which the glucose of the patient will reach a threshold value predicted. Based upon the predicted glucose information, the measurement system can take a sample at the corresponding time. A model can also be used to determine an expected glucose value at various times, responding to changes in patient or environmental conditions such as infusion rates, and indicate a sample be taken when the expected glucose value reaches a threshold or predetermined limit.

The preceding modeling methods can be updated, trained or adjusted by using actual values obtained by the measurement system. For example, the actual measured glucose value can be compared to the value predicted by a physiologic model and a variety of model parameters adjusted as needed. Experience with the response of a particular patient can thereby be used to further improve the safety of the system while also reducing unnecessarily frequent sampling.

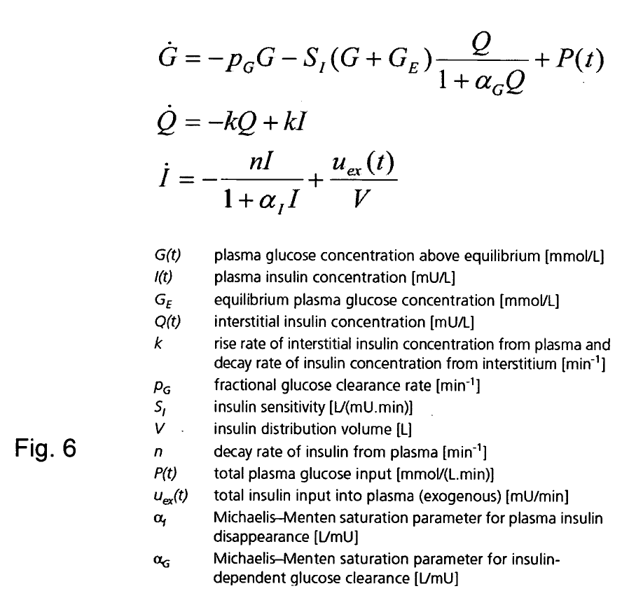

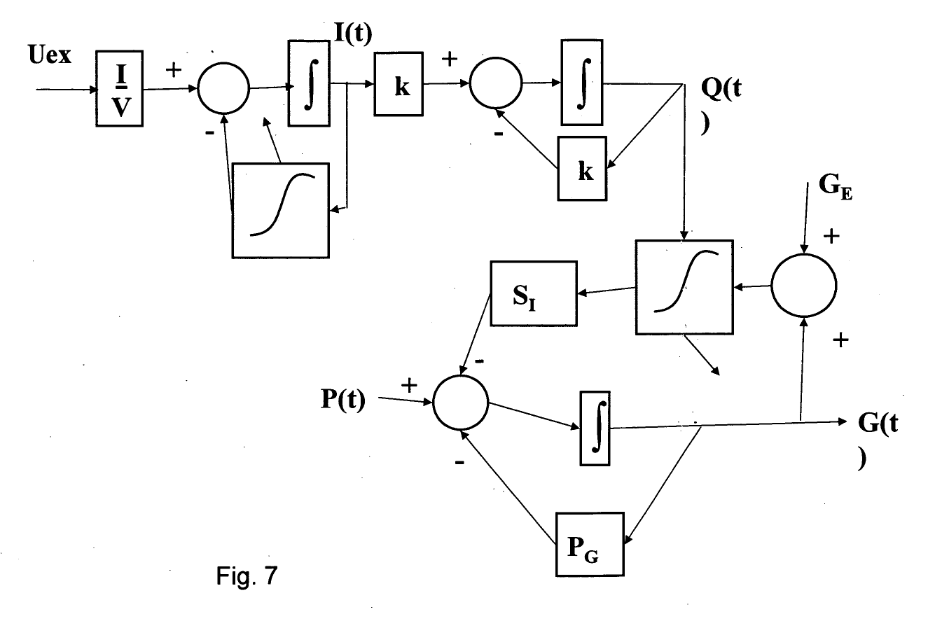

Example Embodiment. FIG. 6 presents the equations governing the Chase et al. model as well as the input parameters. Chase et al. use a model loosely based on Bergman's minimal model with additional non-linear terms and a grouped term for insulin sensitivity. The model effectively incorporates the effect of previously infused insulin with an accounting for the effective life of insulin in the system. The patient's endogenous glucose clearance and insulin sensitivity are represented in the model. The model also used Michaelis-Menton functions to model saturation kinetics associated with insulin disappearance and insulin-dependent glucose clearance. The P(t) term can also be based upon glucose appearance from enteral nutrition via feeding tubes or by direct glucose administration. FIG. 7 is a state diagram of the Chase model showing the key inputs and relationships of the model.

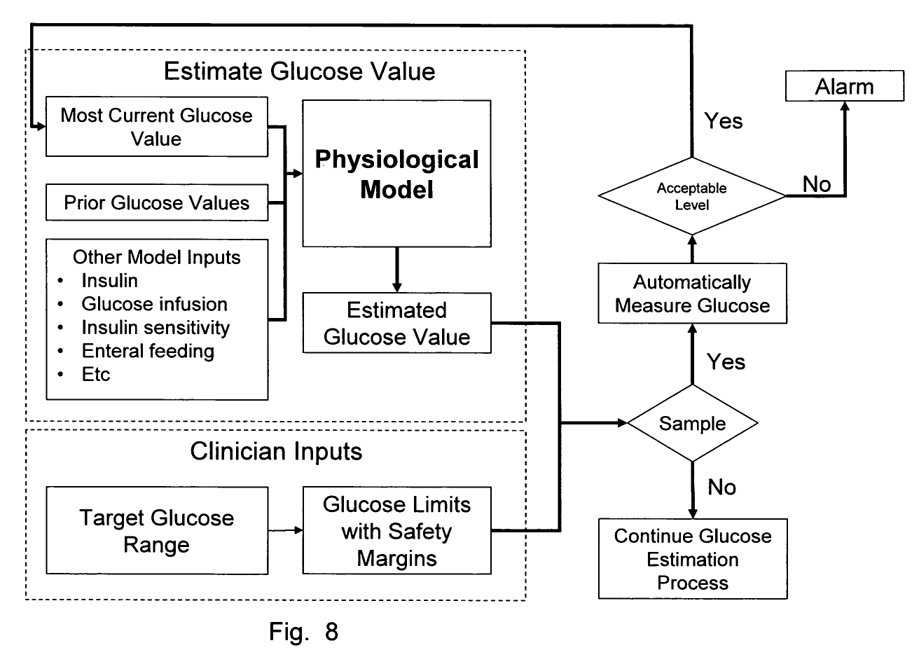

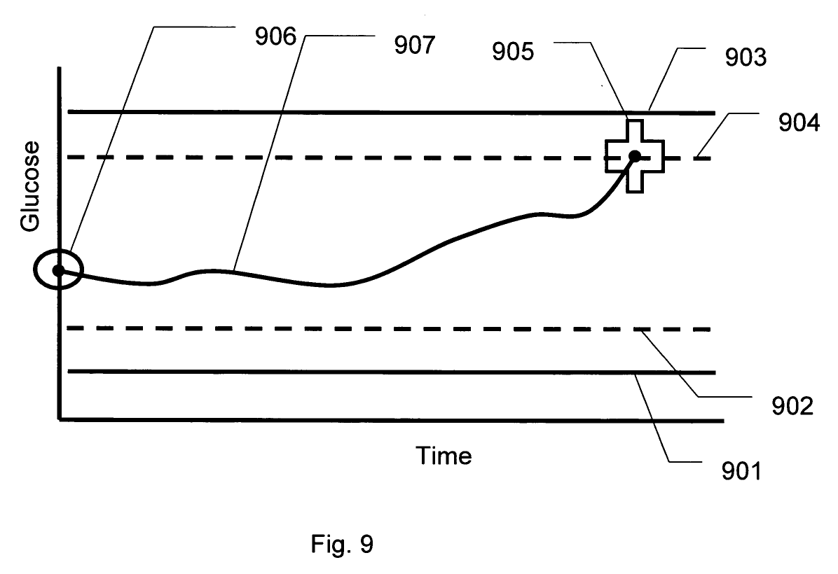

FIG. 8 is a schematic illustration of an example of using a physiological model such as the Chase model as an estimator of glucose concentration and the use of such an estimate to determine the next measurement time. In practice, a clinician can define a desired glucose target range. The system or clinician can apply appropriate safety margins to assure the earliest possible warning that a patient is approaching the target range or is out of the target range. The safety adjusted target range can then be used to determine the need for an automated glucose measurement. The estimated glucose value at a given time point can be determined by a variety of inputs, including prior glucose values, insulin infusion rates, glucose administration and enteral feeding rates. The physiological model or other estimator type models then estimate the glucose concentration. At the point in time that the estimated glucose concentration is no longer in the safety adjusted target range, an automated glucose measurement is made. The measured glucose is used as an input to the estimator model and any model updates made. If the measured glucose value is within the target range the estimation of future glucose values is continued and the process repeated. If the value is outside the target range an indicator or alarm can be generated so that the clinician can address the situation. FIG. 9 shows a graphical representation of the automated determination of the next measurement. The graph shows the last known measurement result 906 and a curve 907 representing the estimated glucose values over time. The estimated value can be predicted into the future based on just the last measurement and the model, or can be determined real time based upon changing current conditions. For example, if following the last measurement the insulin infusion rate were decreased the model can account for that change and re-estimate the glucose value based upon current information. The graph also shows the target glucose ranges (high 903, low 901) with safety margins (high 904, low 902). The safety margins can be symmetrically or asymmetrically set, e.g., some clinicians might view hypoglycemia as a more dangerous condition. At the time point 905 where the estimated glucose concentration intersects with the safety adjusted target glucose range, a sample measurement is automatically obtained.

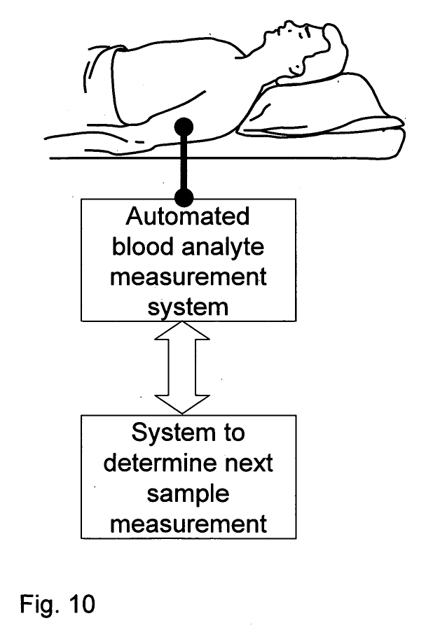

Example Embodiment. FIG. 10 shows a generic embodiment of the system. The operational implementation of the system requires interaction with the patient for the procurement of a blood measurement. This measurement value is then communicated via a variety of possible means to the system that determines the time for the next measurement.

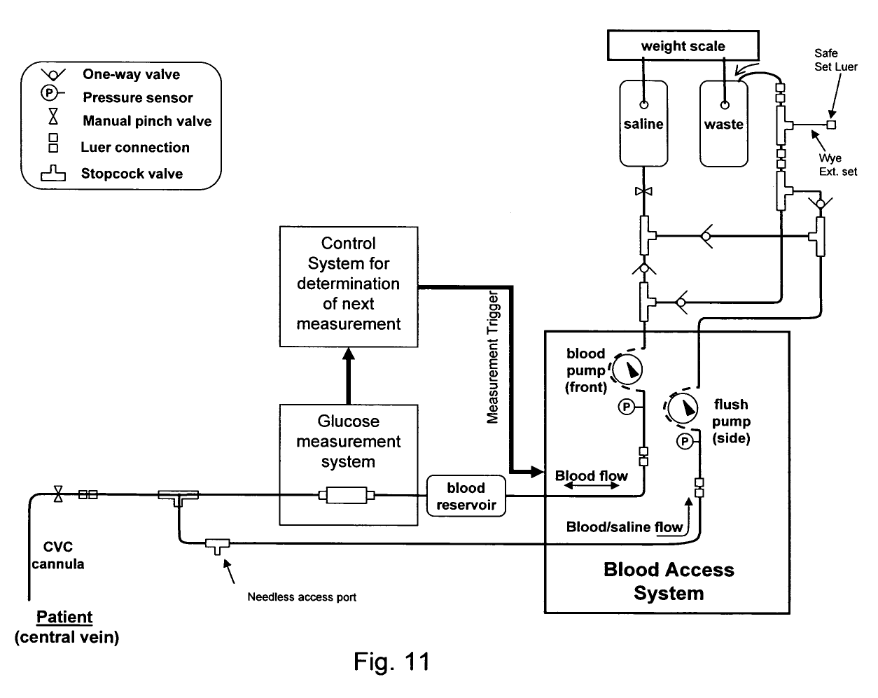

Example Embodiment. FIG. 11 shows an example system in operation on an automated blood removal system. In operation the module labeled “control system for determination of next measurement” initiates the procurement of a glucose measurement. The blood access system initiates blood sample procurement. The blood is presented to the glucose measurement system and a glucose value obtained. The glucose value or related information is communicated to the control system and the time for the next sample determined. The exact methods used for sample procurement can include a manual sample, noninvasive sample, indwelling measurements, or invasive measurement methods. The glucose measurement methods can include existing enzymatic or electrochemical techniques as well as optical measurement methods.

The particular sizes and equipment discussed above are cited merely to illustrate particular embodiments of the invention. It is contemplated that the use of the invention can involve components having different sizes and characteristics. It is intended that the scope of the invention be defined by the claims appended hereto.

What is claimed is:

1. A method of measuring an analyte in a patient, comprising:
a. Measuring the value of the analyte at a first time;
b. Measuring the value of the analyte at a second time;
c. Where the second time is determined from at least one patient condition, at least one environmental condition, or a combination thereof.

2. A method as in claim 1, wherein the second time is determined from a comparison of the value measured at the first time and a threshold value.

3. A method as in claim 2, wherein the elapsed time between the first time and the second time is less for a small difference between the first measured value and the threshold than for a larger difference between the first measured value and the threshold.

4. A method as in claim 1, wherein the analyte is glucose.

5. A method as in claim 1, wherein measuring the value of the analyte comprises using an automated measurement system to measure the value of the analyte.

6. A method of measuring an analyte in a patient, comprising:
a. Measuring the value of the analyte at a plurality of times, with each pair of successive measurements separated by a time interval;
b. Wherein the time intervals are not all the same duration;
c. And wherein at least one time interval is determined from at least one patient condition, or at least one environmental condition, or a combination thereof.

7. A method as in claim 6, wherein at least one time interval is determined by predicting an duration where the value would reach a threshold value, where the prediction is based on one or more preceding measurements and one of: one or more patient conditions, one or more environmental conditions, or a combination thereof; and setting the interval based on the predicted duration.

8. A method as in claim 7, wherein the prediction is based on a linear extrapolation of two or more previous measurements.

9. A method as in claim 7, wherein the prediction is based on a nonlinear curve fitting of three or more previous measurements.

10. A method as in claim 7, wherein the prediction is based on a physiological model of the patient and on at least one preceding measurement.

11. A method as in claim 6, wherein a substance is infused into the patient, and wherein at least one time interval is determined from the nature of the infusate and the rate of infusion.

12. A method as in claim 6, wherein the analyte is glucose, and wherein glucose is infused into the patient, and wherein at least one time interval is determined from information related to the rate of glucose infusion.

13. A method as in claim 6, wherein the analyte is glucose, and wherein insulin is infused into the patient, and wherein at least one time interval is determined from information related to the rate of insulin infusion.

14. A method as in claim 6, wherein at least one time interval is determined by determining whether a change in a patient condition, a change in an environmental condition, or a combination thereof, indicates a measurement should be made.

15. A method as in claim 14, wherein determining whether a change in patient condition, a change in environmental condition, or a combination thereof, comprises applying a physiologic model to the patient's condition, the environmental condition, or a combination thereof, and, if the physiological model indicates a glucose value that approaches a threshold value, then indicating that a measurement should be made.

16. A method as in claim 15, wherein the physiologic model comprises: (a) a model based on the interactions illustrated in the Netter diagram, (b) an AIDA model, (c) a Chase model, (d) a Bergman model, (e) a compartment model with differential equations, (f) an insulin pharmacokinetics and distribution model, (g) a glucose pharmacokinetics and distribution model, (h) a meal model, (i) a glucose/insulin pharmacodynamic model, and (j) an insulin secretion and kinetics model, or (k) a combination of two or more of the preceding.

17. A method as in claim 6, wherein at least one time interval is determined by applying a physiologic model to the patient's condition, environmental condition, or a combination thereof after the preceding measurement, and determining a duration for the time interval, and applying the model again after a change in the patient's condition or environmental condition to determine an updated duration for the time interval, and indicating that a measurement be made after the updated duration.

18. A method as in claim 6, wherein the at least one time interval is determined from a combination of patient condition, environmental condition, or a combination thereof, and previous measurement values.

19. A method as in claim 15, wherein the physiologic model comprises information concerning preceding measured values in relation to patient condition, environmental condition, or a combination thereof.

20. A method as in claim 17, wherein the physiologic model comprises information concerning preceding measured values in relation to patient condition, environmental condition, or a combination thereof.

21. A method as in claim 6, wherein measuring the value of the analyte comprises using an automated measurement system to measure the value of the analyte.

22. A method as in claim 21, wherein measuring the value of the analyte comprises causing the automated measurement system to withdraw a sample of bodily fluid from the patient, measuring the analyte in at least a first portion of the sample, and returning at least a second portion of the sample to the patient.

23. A method as in claim 22, wherein the bodily fluid is blood, and the analyte is glucose, and wherein measuring the value of the analyte comprises determining the response of the first portion to incident radiation, and determining the analyte measurement from the determined response.

24. A method as in claim 21, wherein the bodily fluid is blood, and the analyte is glucose, and wherein the first portion comprises a portion of the blood sample that has substantially all the red blood cells removed.

25. A method as in claim 21, wherein measuring the value of the analyte comprises measuring the analyte with a chemical sensor.

26. A method as in claim 1, wherein the analyte is glucose concentration in blood, and wherein measuring the value of the analyte at a second time comprises withdrawing a blood sample from the patient using an automated system, determining the response of a portion of the blood sample to incident radiation, determining the glucose concentration in the blood sample from the determined response, and infusing at least a portion of the blood sample into the patient.

27. A method as in claim 1, wherein the analyte is glucose concentration in blood, and wherein measuring the value of the analyte at a second time comprises withdrawing a blood sample from the patient, producing a first portion of the blood sample having substantially no red blood cells, and measuring the glucose in the first portion.

28. A method as in claim 1, wherein the analyte is glucose concentration in blood, and wherein measuring the value of the analyte at a second time comprises withdrawing a blood sample from the patient, and measuring the glucose in the in the blood sample using a chemical sensor.

29. An apparatus for measuring the value of an analyte at a plurality of times, comprising:
a. A fluid access system, adapted to withdraw a sample of a bodily fluid from a patient;
b. An analyte measurement system, adapted to measure the value of an analyte in a sample withdrawn from the patient by the fluid access system;
c. A controller, adapted to respond to a patient condition, an environment condition, or a combination thereof, and to cause the fluid access system to withdraw a sample for measurement by the analyte measurement system.

30. An apparatus as in claim 29, wherein the controller determines a time interval from a first sample withdrawal to a second sample withdrawal based on a patient condition, or an environment condition, or a combination thereof.

31. An apparatus as in claim 30, wherein the controller determines a time interval from a comparison of a value of the analyte in connection with the first sample and a threshold value.

32. An apparatus as in claim 31, wherein the controller determines a time interval that has a duration that is less for a small difference between the first measured value and the threshold than for a larger difference between the first measured value and the threshold.

33. An apparatus as in claim 30, wherein the controller predicts a duration until the analyte value will reach a threshold value, where the prediction is based on one or more preceding measurements and one of: one or more patient conditions, one or more environmental conditions, or a combination thereof; and wherein the controller causes the fluid access system to withdraw a sample for measurement based on the predicted duration.

34. An apparatus as in claim 33, wherein the controller predicts a duration by applying a physiologic model based on a patient condition, or an environmental condition, or a combination thereof.

35. An apparatus as in claim 30, wherein the bodily fluid is blood and the analyte is glucose.

36. An apparatus as in claim 31, wherein the bodily fluid is blood, the analyte is glucose, and the model is based on one or more previous glucose values.

37. An apparatus as in claim 34, wherein the model is further based on information related to a rate of glucose infusion.

38. An apparatus as in claim 34, wherein the model is further based on information related to a rate of insulin infusion.

39. An apparatus as in claim 34, wherein the model is further based on the patient's previous response to glucose infusion, or insulin infusion, or a combination thereof.

40. An apparatus as in claim 29, wherein the fluid access system comprises a fluidics system, adapted to remove blood from a body, transport a portion of the removed blood to an analyte measurement system for measurement, infuse a portion of the blood measured by the analyte measurement system back into the patient, flow a maintenance substance to the analyte measurement system without infusing a substantial amount of the maintenance substance into the patient.

41. An apparatus as in claim 29, wherein the fluid access system comprises
a. a blood removal element, adapted to communicate blood with the circulatory system of a patient;
b. a source of maintenance fluid;
c. a waste channel;
d. a fluid control system, in fluid communication with and adapted to control fluid flow among the blood removal element, the analyte measurement system, the source of maintenance fluid; and the waste channel.
